(19) United States
(12) Patent Application Publication
(10) Pub. No.: US 2024/0374504 A1
(43) Pub. Date: Nov. 14, 2024

(54) METHOD TO INHIBIT FUNCTION OF 5-ALPHA REDUCTASE IN THE SCALP SKIN THROUGH COMBINATION OF PLANT-DERIVED EXTRACTS IN A COSMETIC COMPOSITION

(71) Applicant: James Michael Kilgour, Menlo Park, CA (US)

(72) Inventor: James Michael Kilgour, Menlo Park, CA (US)

(21) Appl. No.: 18/650,858

(22) Filed: Apr. 30, 2024

Related U.S. Application Data
(60) Provisional application No. 63/500,977, filed on May 9, 2023.

(51) Int. Cl. A61K 8/9789; A61K 8/14; A61K 8/49; A61K 8/9767; A61K 8/9794; A61Q 7/00

(52) U.S. Cl. CPC A61K 8/9789; A61K 8/14; A61K 8/4953; A61K 8/9767; A61K 8/9794; A61Q 7/00

(57) ABSTRACT

Method to inhibit the function of 5-alpha reductase in the skin of the scalp through the combination of plant-derived extracts in a cosmetic preparation for the scalp is disclosed. The addition of eight plant derived botanical extracts allows the cosmetic preparation to specifically treat androgen-mediated hair loss within the scalp.

### CROSS REFERENCE TO RELATED APPLICATIONS

The present application claims priority to U.S. Provisional Patent Application Ser. No. 63/500,977 filed on May 9, 2023, the contents of which are incorporated herein by reference.

### FIELD OF THE INVENTION

The present invention relates to a method of applying a cosmetic composition to the scalp of a patient for inhibiting function of 5-alpha reductase in the scalp of a patient, therefore preventing hair loss. Further, the present invention relates to a cosmetic composition comprising eight extracts derived from plant as active ingredients for inhibiting function of 5-alpha reductase in the scalp of a patient, therefore preventing hair loss.

### BACKGROUND OF THE INVENTION

Androgen hormones are known to inhibit the growth of hair on the scalp, contributing to hair loss in both men and women. 5-alpha reductase is an enzyme present within the skin which converts the androgen hormone testosterone to dihydrotestosterone (DHT), which is known to be five times more potent than testosterone in activating androgen receptors. Inhibiting the function of 5-alpha reductase is therefore an effective method of reducing the impact of androgen hormones on scalp hair growth. This invention comprises a novel blend of plant and botanical derived extracts which in vivo inhibit 5-alpha reductase. This provides a unique, non-pharmaceutical alternative for the treatment of androgen-mediated hair loss.

One of the plant and botanical derived extracts selected as an active cosmetic ingredient for inhibiting 5-alpha reductase within the skin is caffeine which is a xanthine alkaloid found in non-alcoholic beverages, such as tea, coffee, and cocoa. Caffeine may function as one of the advantageous skin and hair tanning active ingredients.

Another one of the plant and botanical derived extracts selected as an active ingredient for inhibiting 5-alpha reductase within the skin of scalp is turmeric extract that is a Curcuma longa root extract. The turmeric extract is a spice that has anti-inflammatory and antioxidant properties. The turmeric extract contains a chemical called curcumin which can reduce swelling. When used as one of the active ingredients in a cosmetic composition for scalp, the turmeric extract might have a function of regulating the condition of keratinous tissue, therefore regulating hair growth of a patient.

Yet, another one of the plant and botanical derived extracts selected as an active ingredient for inhibiting 5-alpha reductase within the skin of scalp is amla extract that is a Phyllanthus emblica fruit extract. The amla is a sour fruit rich in Vitamin C, antioxidants, and fiber. The amla extract has anti-oxidation, anti-inflammatory, and anti-hyperlipidemic properties. It is also known that applying amla oil to scalp may help with hair loss.

Another one of the plant and botanical derived extracts selected as an active ingredient for inhibiting 5-alpha reductase within the skin of scalp is saw palmetto extract that is Serenoa serrulata fruit extract. The saw palmetto is a tree extract that helps to prevent testosterone from converting to DHT. Thus, the saw palmetto extract may help to prevent hair loss and regulate testosterone levels.

Another one of the plant and botanical derived extracts selected as an active ingredient for inhibiting 5-alpha reductase within the skin of scalp is rosemary extract that is Rosmarinus officinalis leaf extract. The rosemary extract is a natural extract of the herb rosemary, which has anti-inflammatory and circulation-improving properties. It provides help in supporting hair growth and preventing hair loss by stimulating blood flow to the scalp and stimulating nerve growth.

Another one of the plant and botanical derived extracts selected as an active ingredient for inhibiting 5-alpha reductase within the skin of scalp is Ginseng extract derived from Panax ginseng root extract. Ginseng herb contains antioxidants and anti-inflammatory compounds that offer benefits for brain health, immune function, blood sugar control, and more.

Another one of the plant and botanical derived extracts selected as an active ingredient for inhibiting 5-alpha reductase within the skin of scalp is Pumpkin Seed extract that is Cucurbita pepo extract. The pumpkin seed extract contains fatty acids and phytosterols, which is a plant compound that interferes with cholesterol absorption in the intestine. The pumpkin seed extract may help with prostate health, overactive bladder, and hair growth.

Finally, another one of the plant and botanical derived extracts selected as an active ingredient for inhibiting 5-alpha reductase within the skin of scalp is French Maritime Pine Tree extract derived from Pinus pinaster bark extract. The French Maritime Pine tree extract contains chemicals that may improves blood flow, stimulate the immune system, reduce swelling, prevent infections, have antioxidant effects, and help to increase microcirculation in the skin.

The above-mentioned plant and botanical derived extracts are toxicologically acceptable, readily tolerated by the skin, stable in conventional cosmetic formulations or homecare product formulations, inexpensive to prepare, easy to formulate and the compounds need to be active at different pH-values.

### SUMMARY OF THE INVENTION

An objective of the present invention is to provide a method for inhibiting function of 5-alpha reductase in a scalp skin of a patient. The method includes topically applying to the scalp skin with a cosmetic composition made of plant and botanical derived extracts as active ingredients. The active ingredients include Caffeine (Caffeine), Turmeric extract (Curcuma longa root extract), Amla extract (Phyllanthus emblica fruit extract), Saw Palmetto extract (Serenoa serrulata fruit extract), Rosemary extract (Rosmarinus officinalis leaf extract), Ginseng extract (Panax ginseng root extract), Pumpkin Seed extract (Cucurbita pepo extract), and French Maritime Pine Tree extract (Pinus pinaster bark extract).

Another objective of the present invention is to provide a composition for inhibiting the function of 5-alpha reductase in the scalp skin of a patient. The active ingredients in the composition include Caffeine (Caffeine), Turmeric extract (Curcuma longa root extract), Amla extract (Phyllanthus emblica fruit extract), Saw Palmetto extract (Serenoa serrulata fruit extract), Rosemary extract (Rosmarinus officinalis leaf extract), Ginseng extract (Panax ginseng root extract), Pumpkin Seed extract (Cucurbita pepo extract), and French Maritime Pine Tree extract (Pinus pinaster bark extract).

### DETAILED DESCRIPTION OF THE INVENTION

As stated above, androgen hormones are known to inhibit the growth of terminal hairs on the human scalp. The potency of the androgen hormone testosterone is greatly enhanced through the conversion of testosterone to DHT by the enzyme 5-alpha reductase which is present within the skin.

This process of combining a unique blend of plant and botanical derived extracts, named below, functions to neutralize the function of 5-alpha reductase more effectively within the site of the scalp. This provides a naturally derived, pharmaceutical alternative to existing methods of inhibiting 5-alpha reductase in vivo.

The plant and botanical extracts blended within this invention are as follows (with INCI designation contained within parentheses): Caffeine (Caffeine), Turmeric extract (Curcuma longa root extract), Amla extract (Phyllanthus emblica fruit extract), Saw Palmetto extract (Serenoa serrulata fruit extract), Rosemary extract (Rosmarinus officinalis leaf extract), Ginseng extract (Panax ginseng root extract), Pumpkin Seed extract (Cucurbita pepo extract), and French Maritime Pine Tree extract (Pinus pinaster bark extract).

The present invention is an improvement on what currently exists. While cosmetic preparations of the individual plant extracts listed above exist for application to the skin, no current formulation combines all eight of these extracts together to specifically treat androgen-mediated hair loss on the scalp. The lack of combining these eight plant extracts together into one single cosmetic preparation results in an inferior treatment of androgen-mediated scalp hair loss. The addition of these eight plant extracts allows the cosmetic preparation to specifically treat androgen mediated hair loss within the scalp.

The active ingredients in the cosmetic composition of the present invention includes: 1. Caffeine (Caffeine), 2. Turmeric extract (Curcuma longa root extract), 3. Amla extract (Phyllanthus emblica fruit extract), 4. Saw Palmetto extract (Serenoa serrulata fruit extract), 5. Rosemary extract (Rosmarinus officinalis leaf extract), 6. Ginseng extract (Panax ginseng root extract), 7. Pumpkin Seed extract (Cucurbita pepo extract), and 8. French Maritime Pine Tree extract (Pinus pinaster bark extract),

The eight plant-derived extracts listed above are combined to form the cosmetic preparation for the scalp. The process of combining the eight plant extracts results in a unique and novel cosmetic preparation designed to specially treat androgen-mediated hair loss within the scalp. This results in a cosmetic preparation uniquely able to target this common form of hair loss without the need for a pharmaceutical preparation.

According to one embodiment of the present invention, the necessary elements are the combination of the eight plant extracts listed above. A cosmetic preparation for treating the hair loss on the human scalps is conducted by combining the eight active ingredients derived from the plant-derived extracts alongside a suitable vehicle. No specific claim is made as to the relative concentrations of the eight plant extracts included within the blend.

Yet, according to another embodiment, a liposome is used to incorporate one or more of the active ingredients to form liposomal encapsulated active ingredient or ingredients before being mixed with rest of the active ingredients. A preparation procedure is carried out by the following steps: 1. weigh all the ingredients comprising a vehicle, except the ingredients required for liposomal encapsulation, into a container, heat the mixture to 60° C. with gentle mixing, and blend well using a stick blender until all ingredients are fully dissolved; 2. cool the mixture to 40C with gentle mixing; 3. mix the ingredients which require liposomal encapsulation with the unloaded liposome in a separate beaker, add to the above mixture, and mix well until uniform; 4. repeat for all ingredients which require liposomal encapsulation; and 5. test pH with the pH meter, and add 50% citric acid solution or 50% sodium hydroxide solution drop by drop to adjust the pH (if needed).

For the purposes of effectively preventing androgen-mediated hair loss, the cosmetic composition would be applied at least once daily directly to the scalp skin.

What is claimed is:

1. A method for inhibiting function of 5-alpha reductase in a scalp skin of a patient, the method comprising topically applying to the scalp skin with a cosmetic composition including following active ingredients:
Caffeine (Caffeine),
Turmeric extract (Curcuma longa root extract),
Amla extract (Phyllanthus emblica fruit extract),
Saw Palmetto extract (Serenoa serrulata fruit extract),
Rosemary extract (Rosmarinus officinalis leaf extract),
Ginseng extract (Panax ginseng root extract),
Pumpkin Seed extract (Cucurbita pepo extract), and
French Maritime Pine Tree extract (Pinus pinaster bark extract).

2. The method of claim 1, wherein the composition is prepared by combining all the ingredients in a vehicle.

3. The method of claim 1, wherein the composition further includes a liposome.

4. The method of claim 3, wherein the composition is prepared by following steps:
a. providing a liposome,
b. selecting one or more of the active ingredients for liposomal encapsulation,
c. blending the not selected active ingredients in a vehicle until forming a uniform mixture,
d. mixing each of the selected active ingredients with the unloaded liposome in a separate vessel before being added to the above mixture to mix until uniform, and
e. adjusting pH using 50% citric acid solution or 50% sodium hydroxide solution.

5. The method of claim 1, wherein the topical application to the scalp skin with the cosmetic composition is performed chronically at least once daily.

6. A cosmetic composition for inhibiting function of 5-alpha reductase in a scalp skin, the composition comprising following active ingredients:
Caffeine (Caffeine),
Turmeric extract (Curcuma longa root extract),
Amla extract (Phyllanthus emblica fruit extract),
Saw Palmetto extract (Serenoa serrulata fruit extract),
Rosemary extract (Rosmarinus officinalis leaf extract),
Ginseng extract (Panax ginseng root extract),
Pumpkin Seed extract (Cucurbita pepo extract), and
French Maritime Pine Tree extract (Pinus pinaster bark extract).

7. The composition of claim 6 further comprising a liposome.
